(19) United States
(12) Patent Application Publication
(10) Pub. No.: US 2021/0259978 A1
(43) Pub. Date: Aug. 26, 2021

(54) LUBRICATION UNIT AND METHOD OF LUBRICATING ENCAPSULATED SOFT GELATIN CAPSULE

(71) Applicant: Strides Pharma Science Limited, Bangalore (IN)

(72) Inventors: Ramchandragowda Thejaswi BUKUNAKERE, Bangalore (IN); Rahman HAFEEZUR, Bangalore (IN)

(21) Appl. No.: 17/255,923

(22) PCT Filed: Jun. 26, 2019
(86) PCT No.: PCT/IN2019/050480

§ 371 Date: Dec. 23, 2020

(30) Foreign Application Priority Data
Jun. 26, 2018 (IN) 201841021787

(51) Int. Cl. A61K 9/48; B65B 5/06

(52) U.S. Cl. CPC A61K 9/4833; A61K 9/4866; B65B 5/06; A61K 9/4825; A61K 9/4858

(57) ABSTRACT

The present invention discloses a lubricating unit/apparatus/system and to the method of lubricating encapsulated soft gelatine capsules and, more particularly, to soft gelatin and non-gelatin capsules to overcome the problem of clumping and nesting of the soft gelatin capsules.

### FIELD OF THE INVENTION

The present invention relates to a lubricating unit/apparatus/system and to the method of lubricating encapsulated soft gelatin capsules and, more particularly, to soft gelatin and non-gelatin capsules to overcome the problem of clumping and nesting of the soft gelatin capsules.

### BACKGROUND OF THE INVENTION

The art of encapsulation has been known for many years, particularly for the production of unit dosage forms containing various pharmaceutical products. Normally, such pharmaceutical capsules are composed of gelatin or some modification thereof, which is fabricated essentially into two different forms, namely, the so-called hard gelatin capsule and the soft gelatin capsule.

It is also known that conventional soft gelatin capsules are a preferred form of administration for medicaments and similar products; especially liquids, pastes, solids dispersed in liquids, or dry solids. Soft gelatin capsules also possess particular advantages for substances which require total protection from air and light, because the gelatin is completely sealed around the contents. An important example is for the encapsulation of vitamins, which has resulted in high degree of stability thereof.

Soft gelatin capsules, now more commonly known as softgels, have been well known and widely used for many years. Softgels generally comprise an outer shell primarily made of gelatin, a plasticizer, and water, and a fill contained within the shell. The fill may be selected from any of a wide variety of substances that are compatible with the gelatin shell. Softgels are widely used in the pharmaceutical industry as an oral dosage form containing many different types of pharmaceutical and vitamin products. In addition to use as an oral dosage form for drugs and vitamins, soft gelatin capsules or softgels are also designed for use as suppositories for rectal or vaginal use. Other uses are for topical and ophthalmic preparations and the like. The cosmetic industry also uses softgels as a specialized package for various types of perfumes, oils, shampoos, skin creams and the like. Softgels are available in a great variety of sizes and shapes, including round shapes, oval shapes, oblong shapes, tube shapes and other special types of shapes such as stars. The finished capsules or softgels can be made in a variety of colors. Also, opacifiers may be added to the shell.

The process for making softgel capsules includes the step wherein the gelatin shell and the fill material come together to form Softgel capsules. It takes place in a closed environment called clean room where the relative humidity is around 20%. The gelatin shell and fill material are brought together simultaneously in the encapsulation machine.

The process is basically performed as follows: a pump delivers the warm gelatin over two chilled drums which are located at both opposite sides of the machine, through a spreader box that sits over each drum. The warm liquid gelatin flows over the drums and this transforms the liquid gelatin into two solid ribbons of gel. The left and right ribbons pass over rollers which feed them through two die rolls. These die rolls determine the shape and size of softgels and cut the Softgel shell from the ribbons as they turn around.

Simultaneously, a sensitive and high accuracy positive displacement pump delivers the fill material into a heated wedge which sits between rotary dies. This wedge injects the fill material into the die cavities between ribbons just right before the die rolls cut the ribbons and seal the two halves together. Warm just formed softgels slide gently through a chute onto a conveyor belt which carries them to the tumble dryer where cooling and drying process takes place.

In more specific detail, typical soft encapsulation machines form at least two flexible gelatin sheets or ribbons by cooling molten gelatin on separate drums then lubricating and guiding the sheets into communication with each other over co-acting dies while simultaneously dispensing a desired quantity of fill material between the sheets in synch with cavities in the outer surfaces of the dies to produce soft capsules.

The soft capsules produced by the encapsulation machine are transported from the encapsulation machine to a dryer to additionally dry the soft capsules and to make them into final form. The soft capsules are transported from the encapsulation machine to the dryer by a conveyor that extends along the front of the encapsulation machine. After drying, the soft gels are sorted (sized), polished, printed, and inspected for their quality. The soft gels are then packaged into suitable containers, typically of low density polyethylene (LDPE) bags, high density polyethylene (HDPE) bottles, or blisters. The recommended storage conditions for the soft gels include a temperature range of 15-30 C. and a relative humidity of not more than 50%.

U.S. Pat. No. 1,970,396 features a method and machine for producing soft gelatin capsules in an automated process. The method involves the formation of two gelatin sheets or films through the use of a gravity fed spreader box, cooling the liquid gelatin on two separate webs, then lubricating and guiding the two sheets into communication with each other between two co-acting dies while simultaneously dispensing the proper amount of medicine or other filling material between the sheets in registration with half cavities in the outer surface of the dies.

Typical soft encapsulation machines form at least two flexible gelatin sheets or ribbons by cooling molten gelatin on separate drums then lubricating and guiding the sheets into communication with each other over co-acting dies while simultaneously dispensing a desired quantity of fill material between the sheets in synch with cavities in the outer surfaces of the dies to produce soft capsules. The capsules are then separated from the matrix and washed with the solvent to remove the mineral oil lubricant and dried.

U.S. Pat. No. 5,735,105 provides a solvent-free gelatin encapsulation process comprising (a) casting a continuous first gelatin ribbon and a continuous second gelatin ribbon; (b) applying a controlled amount of a food-approved lubricant to a first side of the first gelatin ribbon and to a first side of the second gelatin ribbon; (c) applying a controlled amount of a food-approved lubricant to a second side of the first gelatin ribbon and to a second side of the second gelatin ribbon to give a lubricated first gelatin ribbon and a lubricated second gelatin ribbon; (d) uniting the lubricated first gelatin ribbon and the lubricated second gelatin ribbon to form gel pockets and injecting fill material into the gel pockets to give freshly formed gelatin capsules; (e) finishing the freshly formed gelatin capsules to give finished gelatin capsules; and (f) recovering the finished gelatin capsules.

There are various other patents which describe the production of soft gelatin capsules on industrial scale with a lubricating system in conjunction with encapsulating machine for example in U.S. Pat. Nos. 2,775,080, 4,567,714, 5,074,102 and 5,146,730.

Though improvements are made in the basic machine to aid the operations and enhance the quality of the finished soft gelatin capsules, the pharmaceutical industry still faces the challenges of stability of the finished product during storage such as clumping, nesting, leaking, cross linking, mass migration and such like.

To overcome the clumping or nesting issues, the encapsulated capsules were smeared with oil manually. However, the quantity of oil was not distributed equally on to each capsules.

To ensure the equal distribution of the lubricant on to the finished capsules the present inventors felt a need to provide a lubrication apparatus and a method for lubrication of the encapsulated soft gelatin capsules after printing on the capsule and before packing without manual intervention.

The present invention provides the solution to physical issues such as clumping and nesting of soft gelatin capsules without the need of manual intervention. The process is auto-controlled, manual operations are avoided, allowing a reduction in the total process time.

### SUMMARY OF THE INVENTION

In accordance with the above, the present invention provides automated lubricating apparatus and method of lubricating printed encapsulated soft gelatin capsules before packing such that it overcomes the issues of clumping, nesting during storage.

Accordingly, in an aspect, the present invention provides automated method of evenly lubricating printed encapsulated capsules which comprises;
i. passing the printed encapsulated capsules between the moving media having upper and lower layer;
ii. dripping the required quantity of food grade lubricant/oil on the desired area of moving media and smearing the printed gelatin capsules with said lubricant/oil passing through the media.

Accordingly, the process steps of lubricating the printed encapsulated capsules for even distribution of food approved lubricant/oil on to the capsule comprising;
i. providing food approved lubricant/oil from the oil tank to the manifold nozzles on the flexible pipe connected to the pump of the oil tank;
ii. adjusting the oil flow of each nozzle by flow control valves mounted on each nozzle;
iii. loading the printed encapsulated soft gelatine capsules in the hopper of the machine;
iv. passing the capsules in between the moving media cloth/paper having upper and lower layer through controlled gate of the hopper;
v. lubricating the soft gelatin capsules by control dripping of the said lubricant/oil from the nozzle on to the desired area of the moving media cloth/paper and smearing the gelatin capsules with thin film of said lubricant/oil passing through the media.
vi. inspecting each oil smeared capsule followed by packing in the bottle in the integrated bulk packing line.

In another aspect, the present invention provides automated lubricant apparatus for lubricating the printed encapsulated soft gelatin capsules which comprises;
i. Oil tank with a dosing pump integrated with the machine for providing food approved lubricant/oil to the flexible pipes attached to the pump, on starting the machine;
ii. Flexible pipes connected to the pump for providing lubricant/oil to the nozzles assembled manifold on to the said flexible pipes;
iii. Flow control valves with knobs mounted on the said nozzle to adjust the flow of lubricant/oil on to the media;
iv. Hopper attached to the machine for loading printed encapsulated soft gelatine capsules,
v. Controlled gate attached to the said hopper for passing the capsules between the media;
vi. Media comprising cloth or paper of size ranging from 300-400 mm in width for smearing the passing gelatin capsules with a thin film of lubricant/oil dripped on to the said media;
vii. Camera inspection system for inspecting each oil smeared capsule and integrated bulk packing line to pack the lubricated capsules in the bottle.

### DESCRIPTION OF FIGURES

FIG. 1 depicts the lubricant apparatus with flexible pipes, manifold nozzles, flow control valves and media.

FIG. 2 depicts the oil tank/vessel, dosing pump mounted on said oil tank/vessel.

### DETAILED DESCRIPTION OF THE INVENTION

The invention will now be described with reference to certain preferred and optional embodiments, so that the various aspects therein will be more clearly understood and appreciated.

In an embodiment, the present invention discloses automated method of evenly lubricating printed encapsulated capsules which comprises;
i. passing the printed encapsulated capsules between the moving media having upper and lower layer;
ii. dripping the required quantity of food grade lubricant/oil on the desired area of moving media and smearing the printed gelatin capsules with said lubricant/oil passing through the media.

In another embodiment, the process steps of lubricating the printed encapsulated capsules for even distribution of food approved lubricant/oil on to the capsule comprising;
i. providing food approved lubricant/oil from the oil tank to the manifold nozzles on the flexible pipe connected to the pump of the oil tank;
ii. adjusting the oil flow of each nozzle by flow control valves mounted on each nozzle;
iii. loading the printed encapsulated soft gelatine capsules in the hopper of the machine;
iv. passing the capsules in between the moving media cloth/paper having upper and lower layer through controlled gate of the hopper;
v. lubricating the soft gelatin capsules by control dripping of the said lubricant/oil from the nozzle on to the desired area of the moving media cloth/paper and smearing the gelatin capsules with thin film of said lubricant/oil passing through the media.
vi. inspecting each oil smeared capsule followed by packing in the bottle in the integrated bulk packing line.

In yet another embodiment, the present invention discloses automated lubricant apparatus for lubricating the printed encapsulated soft gelatin capsules which comprises;
i. Oil tank with a dosing pump integrated with the machine for providing food approved lubricant/oil to the flexible pipes attached to the pump, on starting the machine;
ii. Flexible pipes connected to the pump for providing lubricant/oil to the nozzles assembled manifold on to the said flexible pipes;
iii. Flow control valves with knobs mounted on the said nozzle to adjust the flow of lubricant/oil on to the media;
iv. Hopper attached to the machine for loading printed encapsulated soft gelatine capsules,
v. Controlled gate attached to the said hopper for passing the capsules between the media;
vi. Media comprising cloth or paper of size ranging from 300-400 mm in width for smearing the passing gelatin capsules with a thin film of lubricant/oil dripped on to the said media;
vii. Camera inspection system for inspecting each oil smeared capsule and integrated bulk packing line to pack the lubricated capsules in the bottle.

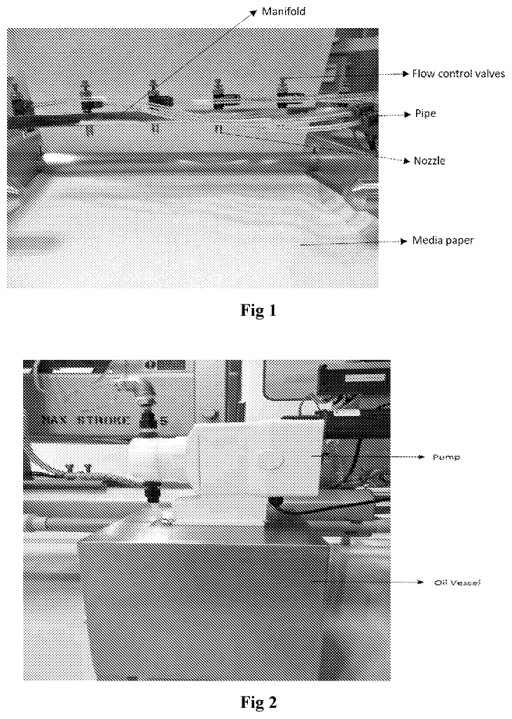

The automated lubricating apparatus and method of lubricating printed encapsulated soft gelatin capsules before packing are described herein with reference to FIGS. 1 and 2. Accordingly, the lubrication apparatus consists of oil tank of approx. 5-7 litre capacity filled with food approved lubricant/oil. Mounted on the oil tank is a dosing pump with knob integrated with the machine which sucks the oil from the oil tank to supply the oil to the manifold nozzles fixed on to the flexible pipes. The flexible pipes are attached to the dosing pump that supplies the oil to the manifold nozzles fixed on to the flexible pipes. The nozzles are fixed with control valves to control the said lubricant/oil flow from each nozzle on to the moving media which is fixed below the nozzles. Hopper with controlled gate is attached to the machine for loading printed encapsulated soft gelatine capsules with hardness of 5-7 newton.

On starting the machine about 0.6-6 litres of food approved lubricant/oil at pressure of 3-4 kg/cm², preferably of 3.5 kg/cm² is pumped from the pump of the oil tank to the nozzles through flexible tubes attached to the said pump. The required quantity of the lubricant/oil is dripped on to the moving media at desired area from the nozzle using control valves. The printed encapsulated soft gelatin capsules fed in the hopper are passed through the media cloth through the controlled gate of the hopper. The thin film of lubricant/oil on the media smears each of the passing capsules with said lubricant/oil through the media. Each oil smeared capsules are inspected by the camera inspection system and then packed in the bottles of the integrated bulk passing line.

The food approved lubricant/oil in the present invention is selected from hydrophilic vehicles such as polyethylene glycols (e.g., PEG400, PEG600), ethoxypolyethylene glycols, propylene glycol and glycerine, Lipophilic vehicles such as free fatty acids, fatty acid esters of hydroxyl compounds such as ethyl alcohol, propylene glycol, glycerin, sorbitol, sucrose, polyethylene glycols, and polyethoxylated fatty acid esters. The fatty acid composition of these esters may vary from short chain (SC, <C8) to medium chain (MC, C8-C10) to long chain (LC, _C12) suited to smear the capsules which will prevent the capsules from clumping, nesting, leaking and such undesirable side effects during storage at a temperature range of 15-30° C. and a relative humidity of not more than 50%.

In an embodiment, the present invention provides said lubricant system in conjunction with conventional soft gelatin encapsulation machine or alternately the soft gelatin encapsulated machine comprising said lubricant system.

In another embodiment, the present invention provides automated lubricant apparatus/system for lubricating encapsulated soft gelatin capsules with food approved lubricant/oil to prevent the capsules from clumping, nesting during storage.

In an embodiment, the automated lubrication of the printed encapsulated soft-gelatin capsules is performed in one-step without manual intervention, the process is auto-controlled, manual operations are avoided allowing a reduction in total process time.

1. An automated method of evenly lubricating printed encapsulated capsules which comprises;
i. passing the printed encapsulated capsules between moving media, the moving media including an upper layer and a lower layer; and
ii. dripping a required quantity of a food grade lubricant/oil on a desired area of the moving media and smearing the printed encapsulated capsules with said lubricant/oil passing through the moving media.

2. An automated method of evenly lubricating printed encapsulated capsules as claimed in claim 1 comprising;
i. providing the food approved lubricant/oil from an oil tank to manifold nozzles on a flexible pipe connected to a pump of the oil tank;
ii. adjusting an oil flow of each nozzle by flow control valves mounted on each nozzle;
iii. loading the printed encapsulated capsules in a hopper;
iv. passing the printed encapsulated capsules in between the upper layer and the lower layer of the moving media through a controlled gate of the hopper, wherein the moving media are cloth or paper;
v. lubricating the printed encapsulated capsules by controlled dripping of the said lubricant/oil from the nozzles on to the desired area of the moving media and smearing the printed encapsulated capsules with a thin film of said lubricant/oil passing through the moving media; and
vi. inspecting each oil smeared printed encapsulated capsule.

3. The automated method of evenly lubricating printed encapsulated capsules as claimed in claim 2, wherein the food approved lubricant/oil is selected from the group consisting of hydrophilic vehicles and Lipophilic vehicles.

4. The automated method of evenly lubricating printed encapsulated capsules as claimed in claim 2, wherein the automated lubrication of the printed encapsulated capsules is performed in one step without manual intervention.

5. An automated lubricant apparatus for lubricating printed encapsulated capsules which comprises;
an oil tank with a dosing pump integrated with the automated lubricant apparatus for providing food approved lubricant/oil on starting the automated lubricant apparatus;
flexible pipes connected to the pump for receiving lubricant/oil from the pump;
a manifold with nozzles for receiving lubricant/oil from the flexible pipes;
flow control valves with knobs mounted on the nozzles to adjust a flow of lubricant/oil;
a hopper attached to the automated lubricant apparatus for loading printed encapsulated capsules,
media comprising cloth or paper of size ranging from 300-400 mm in width for smearing the printed encapsulated capsules with a thin film of lubricant/oil dripped on to the said media from the nozzles to produce lubricated capsules;
a controlled gate attached to the hopper for passing the printed encapsulated capsules between the media; and
a camera inspection system for inspecting each lubricated capsule and an integrated bulk packing line to pack the lubricated capsules in bottles.

6. (canceled)

7. Printed encapsulated capsules which are free from clumping or nesting during storage, wherein the printed encapsulated capsules are obtained by the process as claimed in claim 1.

8. Printed encapsulated capsules which are free from clumping or nesting during storage, wherein the printed encapsulated capsules are obtained by the process as claimed in claim 2.

9. The printed encapsulated capsules of claim 7, wherein the printed encapsulated capsules are gelatin capsules.

10. The printed encapsulated capsules of claim 7, wherein the printed encapsulated capsules are non-gelatin capsules.

11. The automated method of evenly lubricating printed encapsulated capsules as claimed in claim 2, wherein the food approved lubricant/oil is selected from the group consisting of polyethylene glycols, ethoxypolyethylene glycols, propylene glycol, glycerine, free fatty acids, fatty acid esters of hydroxyl compounds, and polyethoxylated fatty acid esters.

12. The automated method of evenly lubricating printed encapsulated capsules as claimed in claim 2, wherein the food approved lubricant/oil is selected from the group consisting of PEG400; PEG600; and fatty acid esters of hydroxy compounds selected from the group consisting of ethyl alcohol, propylene glycol, glycerin, sorbitol, sucrose, and polyethylene glycols.

13. The automated method of evenly lubricating printed encapsulated capsules as claimed in claim 2, further comprising packing the oil smeared printed encapsulated capsules in bottles in an integrated bulk packing line.
